(19) United States
(12) Patent Application Publication
(10) Pub. No.: US 2003/0138856 A1
(43) Pub. Date: Jul. 24, 2003

(54) Process for the measurement of dinophysistoxin and yessotoxin

(76) Inventor: Gian Paolo Rossini, San Lazzaro (IT)

Correspondence Address: McCormick Paulding & Huber, CityPlace II, 185 Asylum Street, Hartford, CT, 06103-4102, US

(21) Appl. No.: 10/312,493

(22) PCT Filed: Jun. 29, 2001
(86) PCT No.: PCT/EP01/07487

(30) Foreign Application Priority Data
Jun. 30, 2000 (IT) MI2000 A 001474

(51) Int. Cl. G01N 33/53; G01N 33/567

(52) U.S. Cl. 435/7.2

(57) ABSTRACT

The present invention relates to a method for the detection, identification and meausrement of toxins belonging to the group of dinophysistoxins and of yessotoxins, based upon the use of in vitro cell cultures.

### FIELD OF THE INVENTION

The technical field of the present invention relates the detection of toxins and their measurement by a system of in vitro cultured cells.

### BACKGROUND ART

The human intoxication caused by ingestion of shellfish or fish contaminated by algae producing toxic substances is a phenomenon which has shown a threatening development in the last years, inasmuch as episodes of intoxication occurs more often and in areas of the planet where they had never been reported in the past.

Several types of intoxication have been described so far, based onto the symptomatology induced in animals and, whenever possible, on the chemical characteristics of causative agents and their mechanism of action. One of these is the Diarrhetic Shellfish Poisoning (DSP) which is due to the ingestion of contaminated molluscs (mainly mussels and scallops) (Yasumoto T., Oshima Y., Yamaguchi M., 1978, Bull. Jpn. Soc. Sci. Fish., 44:1249).

Although lethal outcomes have never been described for DSP intoxication in humans, this can cause serious problems of public health, which are managed following the present rules (in the European Union, Directive CE 91/492/CEE), by an accurate prevention implemented by a continuous monitoring of toxicity of molluscs which, once it is established, implies the prohibition of mollusc harvest and marketing, which lasts until molluscs show significant levels of toxic compounds.

Presently, the measurement of agents classified among DSP toxins can be carried out by chemical (HPLC eventually coupled to Mass Spectrometry) (Quilliam M. A.; J. AOAC Int., 1995, 78:555) and immunological (RIA, ELISA; like those described for instance in EP 509819) methods, which are performed on crude or semi-purified extracts obtained from mollusc tissues, and, in the case of immunological methods, are based on the availability of specific antibodies which have been obtained only for okadaic acid (OA) and for some dinophysistoxins (DTX ).

Chemical methods can yield accurate measurements of most components classified among DSP agents but are not routinely employed in monitoring programmes of toxicity in bivalve molluscs, because the actual toxicity of contaminated material may be underestimated. Indeed, while three groups of chemically distinct components have been classified as DSP toxins (Dinophysistoxins, Yessotoxins and Pectenotoxins) (Yasumoto T., Murata M., 1993, Chem. Rev., 93:1897), the actual human toxicity of every component which can be found in contaminated molluscs has not been firmly established. Furthermore, the type and concentrations of components classified as DSP detectable in molluscs, can change depending on their geographical origin and the time of harvest, so that every sample shows an individual “toxinological profile”.

Moreover, the chemico-physical methods which can be employed for the analysis of each of the three groups of toxins, individually, do not allow the contemporary detection of components belonging to more than one group, which may be present in the same sample.

The detection of DSP toxins can be also carried out by biochemical or cellular methods (functional assays), based onto the measurements of the effects that the toxins can trigger upon association to their specific molecular targets, by modifying their activities, or else based onto the total cytotoxic effect exerted by the toxins on cultured cells capable to sense and react to the toxins. Presently, the biochemical assays are confined to those involving measurements of the inhibition of phosphoprotein phosphatase activity, such as described in WO 96/40983 for measurement of dinophysistoxin 1 and okadaic acid, which are suitable only in the case of toxin functionally related to this latter compound (Tubaro A et al., 1996, Toxicon, 34:743) and displaying the same mechanism of inhibition of that group of enzymes. In the assays based on the use of cultured cells, in turn, the analysis is confined to correlations between the dose of toxin supplied to the system and either the decrease in the number of surviving cells, or the appearance of morphological alterations in affected cells (Fladmark K. E. et al., 1998, Toxicon; 36:1101).

Finally, the third type of detection method involves biological assays capable to detect DSP toxicity. In these assays, the overall effect of the set of components, but not that of individual substances in a complex system, represents the measured parameter. These methods include bioassays of the total toxin pool employing whole living animals (Yasumoto T. et al in: “Seafood Toxins”, Ragelis E. P., Ed., ACS Symposium Series, 262, 1984, pp. 207-214). More specifically, the contaminated material, obtained by extraction of molluscs with organic solvents, is administered to mice, usually by intra-paritoneal injection (ibidem), or else it is added to the culture media of small crustacean, such as D. magna (Vernoux J. P et al., 1993, Food Addit. Contam. 10:603), and the death of the animals is monitored within a fixed time span.

The mouse bioassay described above is the detection method employed in most Countries of the EU and in Japan, to detect total DSP toxicity due to DTX and/or YTX in contaminated molluscs. Indeed the LD₅₀ internationally set for these toxins refer to the mouse bioassay (Yasumoto T et al. in: “Harmful Algae”, B Reguera, J. Blanco, M A Fernandez T. Wyatt, Edd.; Xunta de Galicia and Intergovernmental Oceanographic Commission of UNESCO, 1998, pp 461-464). This assay, however, poses relevant ethical problems, showing an increasing concern in European Countries, whose impact is even more pronounced if one considers that the analysis of a single mollusc sample implies the sacrifice of at least five animals. Furthermore the results of this bioassay may include false positives, probably due to the presence of high levels of fatty acids in the material injected into mice (Quilliam M. A., Wright J. L. C.; in “Manual on Harmful Marine Microalgae”, Hallgraeff G. M., Anderson D. M., Cembella A. D., Edd., IOC Manuals and Guides No. 33. UNESCO 1995, pp.95-111).

### SUMMARY OF THE INVENTION

The present invention relates to a process for the qualitative and quantitative detection of dinophysistoxins and of yessotoxins, based onto the measurement of the amount of the protein called E-cadherin and of the antigens correlated to this protein, ECRA₁₀₀ and ECRA₁₃₅, in an in vitro cellular system. The process, according to the invention, allows the qualitative analysis and the measurement of toxins belonging to the group of yessotoxin and comprising its derivatives and structurally related analogs, such as homoyessotoxin, 45-hydroxyyessotoxin, 44-carboxyyessotoxin, and okadaic acid and its derivatives or structurally related analogs such as dinophysistoxin 1, dinophysistoxin 2 and dinophysistoxin 3. The method comprises the following steps: a) sample preparation; b) incubation of the samples in a cellular system in vitro; c) preparation of cytosoluble extracts from the sample-treated cells and fractionation of the proteins they contain according to the molecular mass of the proteins; d) detection of the E-cadherin antigen and of its related antigens ECRA₁₀₀ and ECRA₁₃₅, by anti-E-cadherin antibodies. The present invention further relates to the use of the process to measure the abovementioned toxins, with the aim to evaluate the contamination of seafood.

In a further embodiment, the invention relates the E-cadherin related antigen ECRA₁₀₀ and the use of the E-cadherin related antigens ECRA₁₀₀ and ECRA₁₃₅ to detect the presence and measure the levels of toxins in a sample.

### BRIEF DESCRIPTION OF THE FIGURES

FIG. 1. Effect of okadaic acid and yessotoxin on E-cadherin and related antigens in MCF-7 cells.

MCF-7 cells were treated with 50 nM okadaic acid, 50 nM yessotoxin, with both agents at a 50 nM final concentration, or with vehicle (control) for 18 hr at 37° C. At the end of the treatment, the cells have been used to prepare cytosoluble extracts and samples corresponding to 50 μg of protein from each extract have been fractionated by SDS-PAGE and subjected to immunoblotting using an anti-E-cadherin antibody. The treatments have been specified on the top of the figure, and the electrophoretic mobilities of the β-galactosidase (118 kDa) and fructose, 6-P kinase (90 kDa) subunits, used as molecular weight markers, have been indicated on the left.

The typical pattern observable after cell treatment with OA, YTX or both, as compared to controls, can be observed: indeed, the appearance of ECRA₁₃₅ can be observed in the lane corresponding to the treatment with OA alone, and is also detectable after the double treatment (okadaic acid+yessotoxin), whereas a notable increase in the immunoreactivity corresponding to ECRA₁₀₀ is observable in the lane corresponding to the treatment with yessotoxin alone and is maintained also after the double treatment.

FIG. 2. Effect of increasing okadaic acid concentrations on the levels of E-cadherin and related antigens in MCF-7 cells.

MCF-7 cells have been treated with the indicated okadaic acid concentrations for 20 hr at 37° C. At the end of the treatment cytosoluble extracts have been obtained, and have been subjected to fractionation by SDS-PAGE and immunoblotting, to detect their content in E-cadherin (filled circles), ECRA₁₃₅ (squares) and ECRA₁₀₀ (triangles), as described in the paragraph “Materials and Methods”.

Values are expressed as percentages of total immunoreactivity (E-cadherin+ECRA₁₃₅+ECRA₁₀₀) in each sample (filled symbols), or as relative total immunoreactivity (Σ), which has been expressed as percentages of the total immunoreactivity of that sample, as compared to that measured in control cells (or the extract obtained from cells which have received vehicle only) (void circles). Data represent means±S.D. from 3-7 separate experiments.

FIG. 3. Effect of increasing yessotoxin concentrations on the levels of E-cadherin and related antigens in MCF-7 cells.

MCF-7 cells have been treated with the indicated yessotoxin concentrations for 20 hr at 37° C. At the end of the treatment cytosoluble extracts have been obtained, and have been subjected to fractionation by SDS-PAGE and immunoblotting, to detect their content in E-cadherin (filled circles) and ECRA₁₀₀ (triangles), as described under “Materials and Methods”.

Values are expressed as percentages of total immunoreactivity (E-cadherin+ECRA₁₃₅+ECRA₁₀₀) in each sample (filled symbols), or as relative total immonoreactivity (Σ), which has been expressed as percentages of the total immunoreactivity of the sample as compared to that measured in control cells (or the extract obtained from cells which have received vehicle only) (void circles). Data represent means±S.D. from 3-7 separate experiments.

FIG. 4. Effect of yessotoxin on the alterations induced by increasing okadaic acid concentrations on the levels of E-cadherin and related antigens in MCF-7 cells. MCF-7 cells have been treated with the indicated okadaic acid concentrations, and either in the presence (solid line) or in the absence (dashed line) of 10 nM yessotoxin for 20 hr at 37° C. At the end of the treatment, cytosoluble extracts have been obtained, and have been subjected to fractionation by SDS-PAGE and immunoblotting, to detect their content in E-cadherin (Panel A), ECRA₁₃₅ (Panel B) and ECRA₁₀₀ (Panel C), and the relative total immunoreactivity (Σ) of the samples (Panel D), as described under “Materials and Methods”.

Values are expressed as percentages of total immunoreactivity (E-cadherin+ECRA₁₃₅+ECRA₁₀₀) in each sample (Panels A-C), or as relative total immonoreactivity (Σ, Panel D), which has been expressed as percentages of the total immunoreactivity of that sample as compared to that measured in control cells (or the extract obtained from cells which have received vehicle only). Data represent means±S.D. from 3 separate experiments.

FIG. 5. Effect of okadaic acid on alterations induced by increasing yessotoxin concentrations on the levels of E-cadherin and related antigens in MCF-7 cells. MCF-7 cells have been treated with the indicated okadaic acid concentrations, and either in the presence (solid line) or in the absence (dashed line) of 50 nM okadaic acid for 20 hr at 37° C. At the end of the treatment, cytosoluble extracts have been obtained, and have been subjected to fractionation by SDS-PAGE and immunoblotting, to detect their content in E-cadherin (Panel A), ECRA₁₃₅ (Panel B) and ECRA₁₀₀ (Panel C), and the relative total immunoreactivity (Σ) of the samples (Panel D), as described under “Materials and Methods”.

Values are expressed as percentages of total immunoreactivity (E-cadherin+ECRA₁₃₅+ECRA₁₀₀) in each sample (Panels A-C), or as relative total immonoreactivity (Σ, Panel D), which has been expressed as percentages of the total immunoreactivity of that sample as compared to that measured in control cells (or the extract obtained from cells which have received vehicle only). Data represent means±S.D. from 3 separate experiments.

FIG. 6. Effect of AO and YTX added to crude extracts prepared from mussel hepatopancreas on E-cadherin and related antigens.

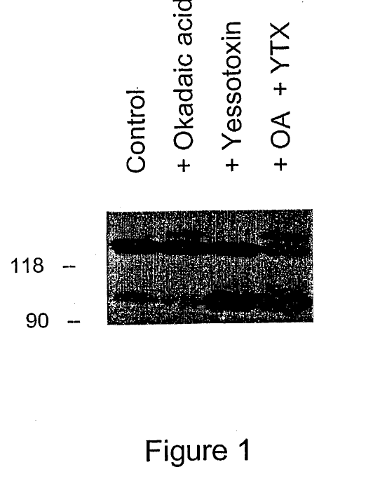

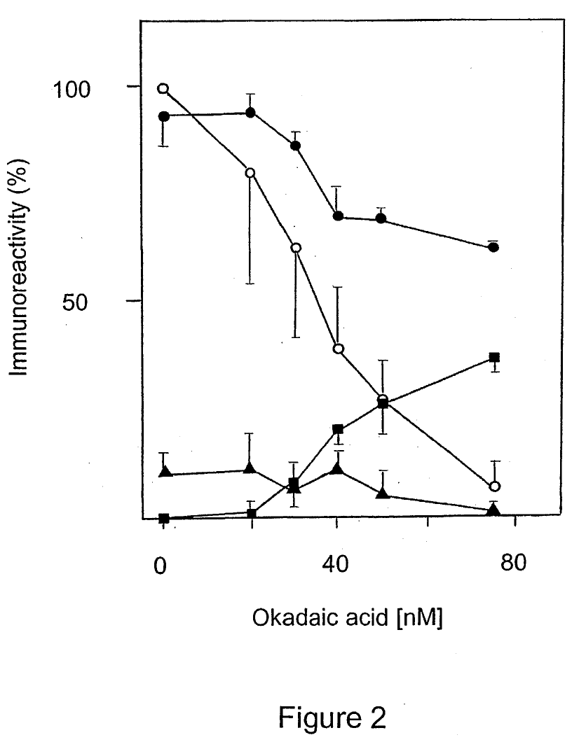

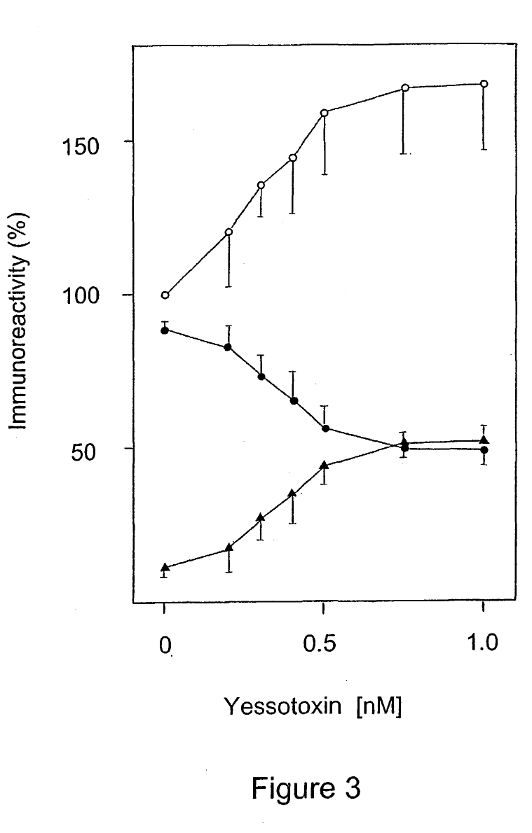

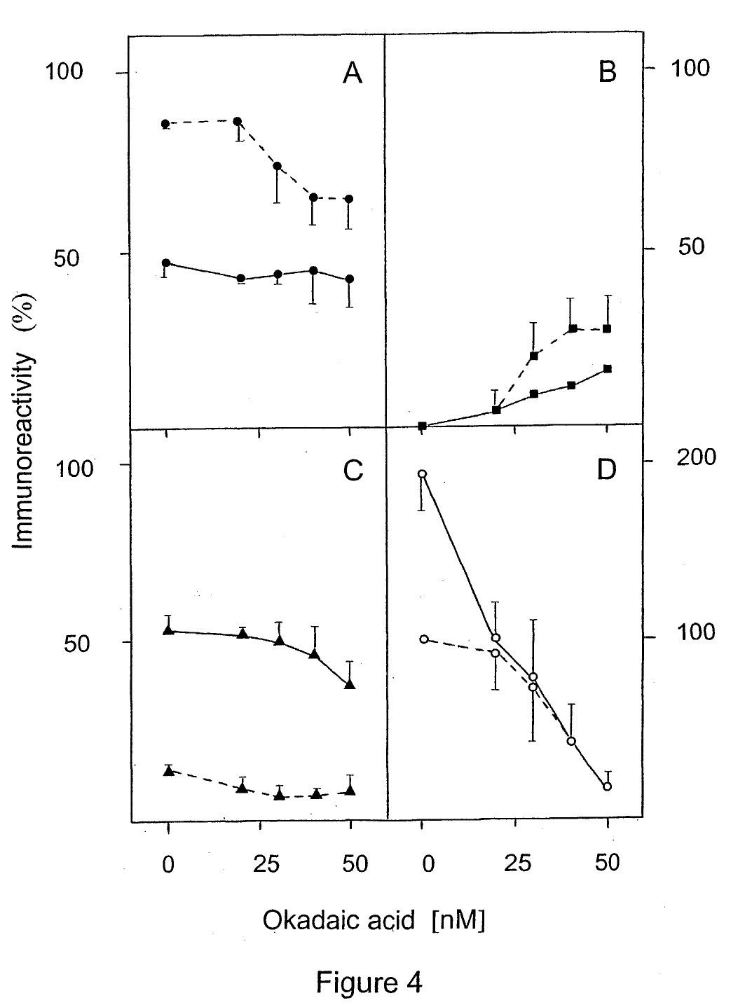

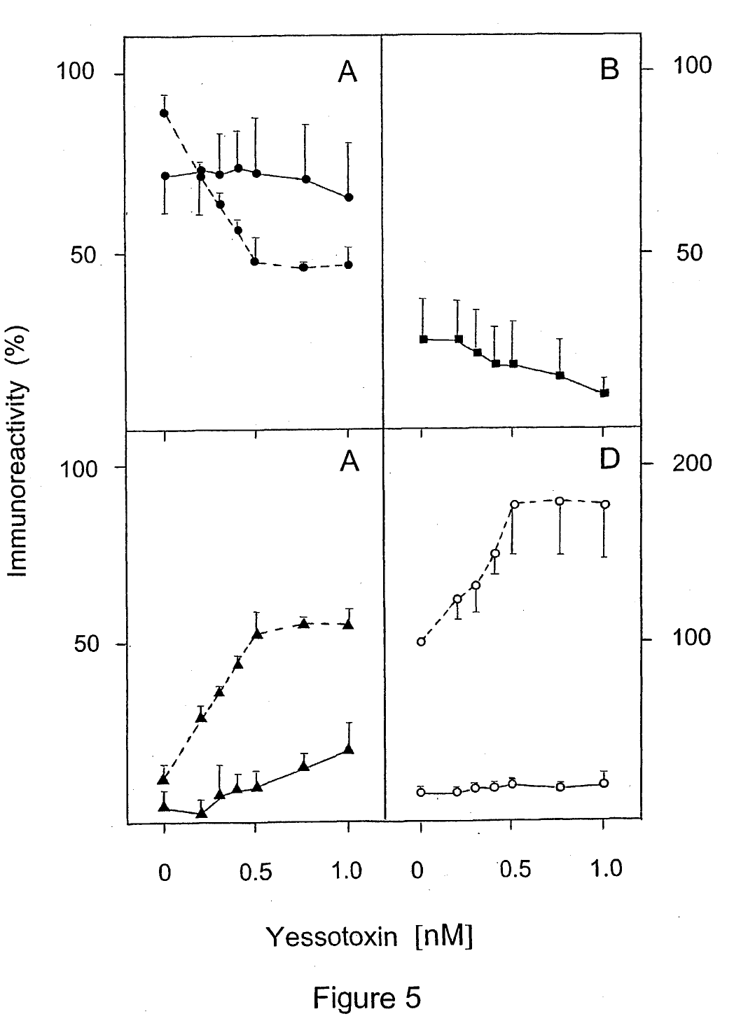

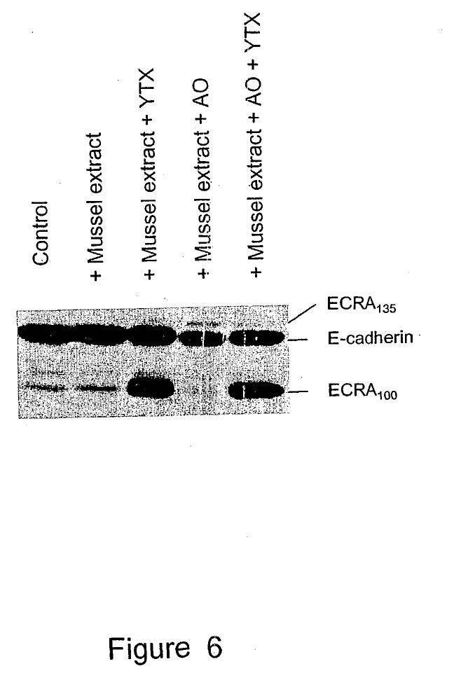

MCF-7 cells have been treated with either the indicated extracts, prepared as described in the text, or vehicle alone (control), for 18 hr at 37° C. At the end of the treatment, cells have been used to prepare cytosoluble extracts and samples corresponding to 50 μg of protein from each extract have been fractionated by SDS-PAGE and subjected to immunoblotting using an anti-E-cadherin antibody. It can be observed that the typical pattern shown in FIG. 1 is also obtained when cells are treated with crude mussel extracts spiked with OA, YTX, or both, as compared to control cells and cells which have been treated with a mussel extract devoid of toxins: in the lane corresponding to the treatment with OA alone, the appearance of ECRA₁₃₅ antigen can be detected, and this antigen is also detected after the double treatment (OA+YTX); the increased levels of the ECRA₁₀₀ antigen, in turn, is detectable in the lane corresponding to the treatment with YTX alone, and is also maintained after the double treatment (OA+YTX).

### DETAILED DESCRIPTION OF THE INVENTION

The method of the present invention relates to a process to detect the presence, to identify the group and to evaluate the amounts of toxins belonging to the group of dinophysistoxins and of yessotoxins, based on the measurements of the intracellular levels of the E-cadherin antigen and of E-cadherin-related antigens (ECRA₁₀₀ and ECRA₁₃₅), in a cellular system.

It has been surprisingly found that the presence of toxins belonging to the classes of dinophysistoxins (Yasumoto T et al., 1993, Chem Rev: 93, 1897) and yessotoxins (Yasumoto T et al. ibid.; Satake et al., 1977, Nat Toxins, 5: 107; Ciminiello et al., 2000, Eur J Org Chem, 291), in an appropriate cellular system, is associated with measurable changes in the cellular levels of E-cadherin and of its related-antigens ECRA₁₀₀ and ECRA₁₃₅.

E-cadherin is a cell adhesion molecule, termed also uvomorulin or L-CAM, or else Cell CAM 120/80, whose human sequence is deposited in the database (SwissProt) under the accession number Z18923. Within the scope of the present invention, E-cadherin refers also to a protein showing a molecular mass of about 126 kDa (126.1±4.1 kDa), in agreement with literature data (Damsky C. H. et al.,1983, Cell, 34:455), as determined by polyacrylamide gel electrophoresis under denaturing and reducing conditions (SDS-PAGE in the presence of β-mercaptoethanol), and recognized by anti-E-cadherin antibodies. Furthermore are defined E-cadherin related antigens the ECRA₁₀₀ and ECRA₁₃₅ antigens (ECRA: E-Cadherin Related Antigens), showing respectively a molecular mass of 100 kDa (101.9±3.2 kDa) and 135 kDa (136.3±3.3), as determined by a series of measurements carried out by SDS-PAGE. Also these antigens are recognized by specific anti-E-cadherin antibodies.

Within the scope of the present invention, a cellular system (or cell system) refers to a cell line, maintained in culture according to methods known to the art, derived either from continuous cell lines or primary cultures, provided that they expresses E-cadherin, preferably the human protein. Preferably, these cell lines are of mammalian origin and even more preferably, these are human cells, such as: Caco-2, A549, BxPc3, MCF-7. In one of the most preferred embodiments, the cellular system is constituted by the MCF-7 cell line (ECACC No: 86012803), cultured following to methods known by one skilled in the art. Are also comprised within the definition of appropriate cell system, cell lines other than human or naturally not expressing the E-cadherin antigen, transfected by DNA sequences encoding for E-cadherin, preferably human E-cadherin.

The toxins detectable and measurable according to the method of the present invention are the ones belonging to the group of dinophysistoxins, preferably represented by dinophysistoxin 1 (DTX1), dinophysistoxin 2 (DTX2), dinophysistoxin 3 (DTX3) and okadaic acid and their derivatives, included toxins structurally related to okadaic acid, as well as those belonging to the group of yessotoxins, such as yessotoxin (YTX), 44-carboxyyessotoxin, homoyessotoxin, 45-hydroxyyessotoxin (in agreement with Yasumoto T et al., 1993, Chem Rev: 93, 1897; Satake et al., 1977, Nat Toxins, 5: 107; Ciminiello et al., 2000, Eur J Org Chem., 291), included toxins structurally related to yessotoxin.

The molecular “patterns” in E-cadherin and immunologically related antigens observed in cellular extracts from cultures treated with various concentrations and combinations of the toxins belonging to the two groups, according to the present invention are summarized in the following table, which reports the relative levels of E-cadherin and immunologically related antigens:

TABLE 1

| | | | | YTX + | YTX + |
|---|---|---|---|---|---|
| Antigens | control^{C} | YTX¹ | DTX² | DTX³ | DTX⁴ |
| E-cadherin* | 85-100 | 40-60 | 60-70 | 40-80 | 40-70 |
| ECRA₁₀₀ * | 0-15 | 40-60 | 0-10 | 10-50 | 20-30 |
| ECRA₁₃₅* | 0 | n.d. | 20-40 | 10-30 | 10-30 |
| Σ^{♦} | 100 | 150-250 | 10-20 | 20-100 | 15-20 |

In this table the levels (*) of immunoreactive antigens have been expressed as percentages, as compared to the total immunoreactivity (E-cadherin+ECRA₁₀₀+ECRA₁₃₅) of the sample. Also indicated are the levels (♦) of relative total immunoreactivity (Σ) expressed as percentages of the total immunoreactivity of the sample compared to the total immunoreactivity in the controls. Values have been obtained at each indicated total toxin concentration: (^{c}) [YTX] <0.2 nM_{E}/[DTX] <25 nM_{E}; (¹) [YTX] ≧0.5 nM_{E}/[DTX] <25 nM_{E}; (²) [ DTX] ≧40 nM_{E}/[YTX] <0.2 nM_{E}; (³) [YTX] 0.2-0.5 nM_{E}/[DTX] 25-40 nM_{E}; (⁴) [DTX] ≧40 nM_{E}/ [YTX] >0.5 nM_{E}.

Thus, the presence of dinophysistoxins and their derivatives or structurally related toxins at concentrations higher than 25 nM_{E} (expressed as concentrations equivalent to okadaic acid concentration) in a cellular system is associated with a typical E-cadherin and related antigens pattern, comprising: a) a decreased E-cadherin immunoreactivity; b) appearance of the E-cadherin related antigen, ECRA₁₃₅; c) decreased relative total immunoreactivity (Σ). The presence of yessotoxin or its derivatives or structurally related toxins at concentrations higher than 0.2 nM_{E} (expressed as concentrations equivalent to yessotoxin) leads to: d) decreased E-cadherin immunoreactivity; e) increase in the E-cadherin related antigen, ECRA₁₀₀; f) undetectable levels of ECRA₁₃₅ as measured for instance, by immunoblotting; g) increased relative total immunoreactivity (Σ).

In one of its embodiments, therefore, the invention yields a process suitable for identifying qualitatively the presence of dinophysistoxins and yessotoxins present either separately, or in combination, in a cellular system, or in a sample whose contamination is to be determined, by the analysis of the appearance or of the increase in ECRA₁₃₅ and ECRA₁₀₀ antigens and the levels of E-cadherin. Preferably, this evaluation is carried out with reference to a control, represented by cells which have not been treated with toxins, or else have been treated with vehicle alone, prepared as the sample.

In summary is therefore comprised within the scope of the present invention, any method useful to detect the levels of E-cadherin and its immunologically related antigens, and/or to visualize each antigen or to identify one or the other typical pattern as described in table 1.

It has been observed that the pattern characteristic for each group of toxins, as summarized in table 1 and shown in FIG. 1, is maintained also when the two groups of toxins are present at the same time in the cell culture, at concentrations higher than 35 nM okadaic acid and 0.4 nM yessotoxin. The present invention, therefore, also relates to a process to detect the presence of each of the two group of toxins present either together or separately in a cellular system or in a sample and to identify the belonging group of the toxin.

In a preferred embodiment the detection or evaluation is performed after immunological recognition of E-cadherin and E-cadherin related antigens performed in cell extracts prepared from the in vitro cell system, with anti-E cadherin antibodies. The analysis is therefore preferably carried out by means of immunological methods, and even more preferably after fractionation of cellular extracts.

Other methods recognizing the molecular species E-cadherin, ECRA₁₃₅ and ECRA₁₀₀, can be used and are comprised within the scope of the present invention, such as biochemical methods to evaluate characteristics other than immunological of the E-cadherin and related antigens: ECRA₁₃₅ and ECRA₁₀₀.

The changes in the levels of E-cadherin, ECRA₁₀₀ and ECRA₁₃₅ are proportional to the toxin concentrations utilized, and indeed, the presence of yessotoxins in a cellular system leads to an increase in ECRA₁₀₀ levels (as observed in extracts prepared from treated cells), reaching 60% of total immunoreactivity (E-cadherin+ECRA₁₃₅ and ECRA₁₀₀), as compared to control cells not treated with the toxin (and under these conditions, the ECRA₁₃₅ antigen is undetectable), whereas the presence of dinophysistoxin in the sample is associated with the appearance of ECRA₁₃₅, whose level can account for up to 40% of total immunoreactivity, as compared to control cells which are not treated with the toxin, and ECRA₁₀₀ is either absent or represent less than 10% of total immunoreactivity. Within the scope of the present invention, the term “total immunoreactivity” indicates the sum of the immunoreactivity due to E-cadherin, ECRA₁₃₅ and ECRA₁₀₀ in any specific sample, measured, for instance, by densitometric analysis of an electropherogram. The term “relative total immunoreactivity” (Σ), in turn, indicates the percentage of the total immunoreactivity of any sample obtained from treated cells, as compared to that of controls, represented by cells treated with vehicle alone or untreated cells.

The present invention, therefore, also relates to a process to detect toxins belonging to the groups of dinophysistoxins and/or yessotoxins, present either separately or combined in a cell culture or in a sample, which comprises: a) the treatment of a cellular system with serial dilutions of the sample and, in parallel, with increasing concentrations of each representative compound belonging to each of the two group of toxins; b) the construction of standard curves of immunoreactivity for the antigens E-cadherin, ECRA₁₃₅, ECRA₁₀₀, and of the relative total immunoreactivity (Σ) for each of the two groups of toxins, where the abovementioned standard curves are constructed preferably using okadaic acid as the reference compound for the class of dinophysistoxins, and yessotoxin as the reference compounds for the class of yessotoxins, at okadaic acid concentrations comprised between 0 and 80 nM for dinophysistoxins, and at yessotoxin concentrations comprised between 0 and 10 nM for yessotoxins; c) the interpolation on the standard curve of the toxin concentration corresponding to value of immunoreactivity of each of the three antigens and to Σ (relative total immunoreactivity), as measured in the sample or in the cellular system.

In this preferred embodiment, E-cadherin, ECRA₁₃₅ and ECRA₁₀₀, are detected in cellular extracts by immunochemical means. They are preferably visualized affer electrophoretic fractionation of cell extracts, transfer of fractionated proteins on a filter and recognition of antigens by anti-E-cadherin antibodies (by immunoblotting or Western blotting techniques). Other immunological methods suitable for selective detection of E-cadherin and related antigens may be also used, and are comprised within the scope of the present invention. Examples of methods which can be used according to the present invention are: RadioImmunoAssays (RIA), Enzyme Linked Immuno Sorbent Assays (ELISA), using monoclonal or polyclonal antibodies carrying or the E-cadherin and/or the ECRA₁₃₅ and/or ECRA₁₀₀ specificity. Other immunological methods known in the art may be also used to identify the E-cadherin and related antigens pattern. Also, any method suitable for selective recognition of ECRA₁₃₅ and ECRA₁₀₀, other than immunological ones, such as those based upon the biochemical characterization of these proteins, are comprised within the present invention. The detection and measurement of the levels of toxins of the classes of dinophysistoxins and yessotoxins, aimed at ascertaining the contamination of foodstuff, according to the process of the present invention, is particularly useful for seafood, particularly for bivalve molluscs, preferably mussels and scallops, intended for human consumption, and can be used as a routinary assay in monitoring programmes aimed at preventing the commercialization of contaminated material.

In one of its preferred embodiments, the process according to the invention provides, therefore, that the sample, preferably constituted by mussel extracts prepared according to standard processes, comprising a homogenization step in acetone and a re-extraction of the residue with diethylether or ethylacetate or dichloro-methane (as reported in the Italian G.U. n° 279, Nov. 29, 1995, D. M. Sanita Jul. 31, 1995, or in the Directive 91/942/EU), here defined as crude extracts, preferably further purified, even more preferably purified by extraction and fractionation by the use of organic solvents, is included in the culture vessels, upon serial dilutions, in the cellular system of choice, as previously defined in details, and incubated with the cells under their culture conditions (for instance 37° C., 5% CO₂) for a time span comprised between 12 and 24 hr, preferably 20 hr. The cultured cells are then washed a few times with isotonic solutions, such as a Phosphate Buffered Saline, and are then lysed according to methods known in-the art, in the presence of ionic detergents such as, for instance, SDS and sodium deoxycholate, optionally associated with non-ionic detergents, such as TritonX-100, and protease inhibitors, such as, for instance, PMSF, aprotinin, etc. The lysis procedure is carried out at 4° C., in order to minimize protein degradation in the samples. Anyway, variations in the cell lysis procedure are easily obtainable by one skilled in the art, by introducing changes in the concentrations of detergents, the time of lysis, etc, provided that conditions of limited proteolysis are used.

The cellular lysate is then partially purified, preferably by centrifugation, and cytosoluble extracts containing the solubilized cellular proteins are prepared. The cytosoluble extract can then be treated with sulfhydryl reducing agents, such as β-mercaptoethanol (5%) and subjected to total denaturation of proteins by heating at 100° C. in the presence of 2% SDS, as well known in the art. Aliquots of cytosoluble extracts corresponding to about 30-50 μg total protein, are then fractionated on the basis of their molecular mass. The fractionation takes place preferably by electrophoresis using polyacrylamide gels at concentrations comprised between 7 and 12%, preferably 10%, of a mixture acrylamide-bisacrylamide, under denaturing conditions (SDS-PAGE), preferably in the presence of sulfhydryl reducing agents, according to Laemmli (Laemmi U. K., 1970, Nature, 227:680). Other established methods suitable for protein fractionation according to their molecular mass, such as capillary electrophoresis (Manabe T., Electrophoresis, 1999, 20:3116), and gel permeation chromatography (Siegel et al. Biochem. Biophys Acta, 1966, 112:346), may also be used.

The fractionated proteins are then transferred on a solid matrix or a filter, for instance a PVDF or nitrocellulose membrane, following methods known in the art, such as the blotting, the electroblotting, or the capillary blotting. Preferably, electroblotting is used and the solid matrix is then employed for the subsequent immunological detection with an anti-E-cadherin antibody, according to the Western Blotting procedure: according to this method, the support matrix carrying the transferred proteins is probed with the primary anti-E-cadherin antibody (monoclonal or polyclonal as such or monospecific) in an appropriate buffer (for instance, TBS: 20 mM Tris-HCl, pH 7.5, 150 mM NaCl) preferably containing CaCl₂ at concentrations comprised between 0.5 and 3 mM, preferably 1 mM. Preferably, anti-E-cadherin antibodies are monoclonal, such as the anti-E-cadherin antibody marketed by Zymed Labs. Inc. (clone HECD-1), used preferably at a concentration comprised between 1-10 μg/ml buffer, preferably 2 μg/ml. In any case, the antibody concentrations and the immunoblotting conditions described here can be easily modified by one skilled in the art, and may be varied according to the antibody used. Other primary antibodies can be used, provided they are anti-E-cadherin antibodies either polyclonal or monospecific or monoclonal. In a preferred embodiment, the monoclonal antibody is specific for an epitope located at the N-terminal part of the E-cadherin molecule.

The primary antibody is then detected by a secondary antibody displaying the appropriate specificity, which is an anti-mouse Ig antibody conjugated with a detectable moiety, for instance horse radish peroxydase (HRP), alkaline phosphatase, biotin, etc. As an alternative, the primary antibody may be directly conjugated with the label molecule. The detection of bands corresponding to E-cadherin, ECRA₁₃₅ and ECRA₁₀₀, is then obtained according to procedures known in the art, for instance by development of a chemoluminescent signal according to the ECL method from Amersham-Pharmacia.

As an alternative to Western blotting procedures, cytosoluble extracts containing proteins labelled for instance with radioactive isotopes (such as ³⁵S or ¹²⁵ I), can be probed with the anti-E-cadherin antibody before being electrophoretically fractionated, for instance according to a RIPA procedure (Radio Immuno Precipitation Assay), and the molecular mass of immunoprecipitated components can be eventually determined by polyacrylamide gel electrophoresis.

The relative levels of E-cadherin and related antigens, ECRA₁₃₅ and ECRA₁₀₀, in the sample, then takes place by exposure of a photographic film, for instance Kodak X-Omat. The elettropherogram is preferably subjected to densitometric scanning, and the absorbance values measured as the area of the peaks of each sample, in arbitrary units, is used to quantify the antigens as detected by the antibody and to determine the values of the total immunoreactivity and of the relative total immunoreactivity (Σ). In the case of a simple qualitative analysis, a visual examination of the autoradiography film allows the direct recognition of the molecular pattern characteristically related to the two groups of toxins. The analyses can be also carried out by a direct comparison between the components from treated cells and those found in controls, such as a cellular system which has not been treated with toxins, or else by the use of molecular mass markers.

The detection procedure according to the invention has the following advantages, as compared to already available methods:

selectivity, due to the measurement of two distinct molecular antigens (ECRA₁₃₅ and ECRA₁₀₀) representing the response to the two different classes of DTX and YTX toxins, respectively, which allows to determine the presence of one of the two classes of toxins, or both, in a sample, and to identify to which group the toxin belongs (qualitative determination)

simplicity in the execution, as both classes of toxins are detected at the same time by a single assay and one antibody,

precision, due to a precise quantification of a measurable parameter, rather than to a subjective and hardly quantifiable evaluation of morphological alterations,

accuracy of measurements, due to the unequivocal characterization of the measured molecular parameters (quantitative determination).

According to a further embodiment, the invention also relates to the molecular antigen ECRA₁₀₀, which is recognized by anti-E-cadherin antibodies and displays a mean molecular mass of 100 kDa (101.9±3.2 kDa) (mean values obtained from measurements performed by SDS-PAGE according to Laemmli U. K. 1970, Nature, 227:680, and by interpolation of the relative electrophoretic mobility of the antigen and those of molecular markers having a known molecular weight, run in parellel lanes, in particular the β-galactosidase (118 kDa) and the fructose,6-P kinase (90 kDa) subunits). The present invention also relates to the use of the antigens E-cadherin, ECRA₁₃₅ and ECRA₁₀₀ to detect the presence, to identify the functional group to which a toxin belongs related to structural similarities, and to measure the levels of toxins, preferably to detect the presence and to measure the levels of toxins of the classes of yessotoxins (YTX) and dinophysistoxins (DTX). Even more preferably such toxin are: yessotoxin, homoyessotoxin, 45-hydroxyyessotoxin, 44-carboxyyessotoxin, dinophysistoxin 1, dinophysistoxin 2, dinophysistoxin 3, okadaic acid, and their derivatives and structurally related analogs.

The invention will be further described in the experimental examples reported below, which do not represent any limitation to the invention.

### EXPERIMENTAL EXAMPLES

### Materials and Methods

Materials. Horse-radish-peroxidase conjugated anti-mouse Ig antibodies and the reagents employed for ECL detection were products from Amersham-Pharmacia. Cell culture media have been obtained from Life Technologies, and plasticsware used for cell culture were from Nunc. Okadaic acid (ammonium salt) was purchased from Alexis Corporation. Yessotoxin has been obtained from the Institute of Environmental Science and Research Limited (New Zealand). The monoclonal anti-E-cadherin antibody (clone HECD-1) employed in these experiments was a product from Zymed Laboratories, Inc. The pre-stained molecular mass markers have been obtained from Sigma. The nitrocellulose membrane “Protran B83” was from Schleicher & Schuell. Cell cultures and treatments.

The MCF-7 cell line employed in these experiments has been obtained from the European Collection of Cell Cultures (ECACC No: 86012803; CB No: CB2705). Cells have been maintained at 37° C. in an atmosphere containing 5% CO₂ in Petri dishes, in a culture medium composed of Dulbecco's modified MEM, containing foetal bovine serum (10%), nonessential aminoacids (1%) and 2 mM glutamine. Cell treatments have been carried out by adding appropriate aliquots of toxins from stock solutions prepared with absolute ethanol, and control cultures received an identical volume of vehicle. Working solutions of the different agents were obtained by serial dilutions of stock solutions, represented by 50 μM okadaic acid and 500 μM yessotoxin, respectively, and were stored at −20° C., in glass vials, protected from light. The ethanol concentrations in culture media has never been higher than 0.5% (v/v), which represents a concentration uncapable to affect the molecular parameters measured in these experiments.

The final concentrations of the agents in the culture vessels and the times of individual treatments are specified in the description of the results we obtained.

Preparation of Cellular Extracts.

The cellular extracts employed in the present analyses have been obtained from the cells adhering to culture vessels at the end of the indicated treatments, and the procedure was carried out at 4° C. Cells were washed three times with 20 mM phosphate buffer, pH 7.4, containing 0.15 M NaCl (PBS) and were then lysed by the addition of 25 μl of lysis buffer/cm² of culture surface area, and by keeping cells in contact with this solution for 10 minutes at 4° C. The lysis buffer was composed of PBS, containing: 0.5% (w/v) Na deoxycholate, 0.1% (w/v) Na dodecylsulfate (SDS), 1% (v/v) Triton X-100, 0.1 mg/ml phenylmethyl sulfonyl fluoride (PMSF). Cellular lysates were then centrifuged for 30 minutes at 16000 xg, and the supernatants of this centrifugation (cytosoluble extracts) were recovered and used to measure their total protein content by a calorimetric method, using bicinchoninic acid (Smith P. K et al., 1985, Anal. Biochem., 150:76). Cytosoluble extracts were then brought to a final 2% SDS, 5% β-mercaptoethanol and 20% glycerol concentration, and were treated for 5 min at 100° C., before being used for protein fractionation by electrophoresis.

Protein Fractionation by Polyacrylamide Gel Electrophoresis in the Presence of SDS (SDS-PAGE) and Immunoblotting Analysis.

Samples, usually containing the same amount of protein, corresponding to about 50 μg, were subjected to polyacrylamide gel electrophoresis in the presence of SDS, according to the procedure by Laemmli (Laemmli U. K. , 1970, Nature, 227:680), using separating gels containing 10% acrylamide monomer. At the end of the electrophoresis (running time about 3 hr at 170 Volts), proteins were electrophoretically transferred to a nitrocellulose membrane (Protran B83), and were then stained with Ponceau S.

Aspecific binding sites on the membrane were then saturated by a 1 hr incubation at room temperature with a solution composed of 20 mM Tris-HCl, pH 7.5 at 25° C., 150 mM NaCl (TBS), containing 3% (w/v) low fat dry milk and 1 mM CaCl₂. The membrane was then incubated with TBS containing 1 % (w/v) low fat dry milk, 1 mM CaCl₂, and 2 μg of anti-E-cadherin antibody/ml buffer, for 1 hr at room temperature. At the end of the incubation the membrane was washed four times for 5 min and then a fifth time for 10 min with TBS containing 0.05% (w/v) Tween 20 (TBS-Tween buffer). The membrane was then incubated with TBS-Tween containing 1% (w/v) low fat dry milk and secondary antibody (horse radish peroxydase conjugated anti-mouse Ig antibody) at a 1:3000 dilution, for 1 hr at room temperature. At the end of this incubation, the membrane was washed as previously described, and the antigens were then detected by the ECL procedure and autoradiography, using Kodak X-Omat films. The electropherogram was then subjected to densitometric scanning, and the absorbance measured from the peak area was recorded and used to quantify the detected antigens.

Reference Compounds Used in These Experiments

Within the embodiment of these experiments, okadaic acid (OA) and yessotoxin (YTX) (see the “Materials” section), have been used as the reference compounds for the classes of dinophysistoxins and yessotoxins, respectively (in agreement with Yasumoto T et al., 1993, Chem Rev: 93, 1897; Satake et al., 1977, Nat Toxins, 5: 107; Ciminiello et al., 2000, Eur J Org Chem., 291). The toxin concentrations used are then expressed as equivalents to those two reference compounds.

### Example 1

### Changes in the Cellular Content of E-Cadherin and Related Antigens ECR₁₃₅ and ECRA₁₀₀, Following Treatment of Cells in Culture with Okadaic Acid (OA) and Yessotoxin (YTX).

MCF-7 cells were treated with 50 nM OA, 50 nM YTX, with both agents at a 50 nM final concentration, or with vehicle (control), for 18 hr at 37° C. At the end of the treatment, the cells were used to prepare cellular extracts, and 50 μg of protein from each sample were fractionated by SDS-PAGE and were subjected to immunoblotting analysis using anti-E-cadherin antibody.

As it is shown in FIG. 1, the immunoreactive material is mainly present as a 126 kDa (126.1±4.1 kDa) molecular mass band, in agreement with data reported in literature (Damsky C. H et al., 1983, Cell, 34:455), both in control and in the treatment with okadaic acid, but in this treatment it is associated with the appearance of ECRA₁₃₅ (136.3±3.3 kDa). After yessotoxin treatment, in turn, most immunoreactivity is associated with the 100 kDa (101.9±3.2 kDa) band, corrisponding to ECRA₁₀₀, and E-cadherin is also detected as a slightly less dark band . Both ECRA₁₃₅ and ECRA₁₀₀, characteristic of OA and YTX treatment, respectively, are detected after the double treatment (OA+YTX), together with the band of native E-cadherin.

The densitometric scanning of electropherograms obtained by similar procedures and under similar experimental conditions has also shown that: after cell treatment with vehicle (control) E-cadherin constitutes the major (92.1±5.4%, n=6) component of total immunoreactivity. Sometimes, and following overexposure of the film, the antigen ECRA₁₀₀ (E-Cadherin Related Antigen) can be observed as a minor component. In cells treated with 50 nM OA, a net decrease in the relative total immunoreactivity (Σ) is detected, reaching 15.4±8.7% of that detected in the extracts prepared from control cells. After OA treatment, E-cadherin still represents the major immunoreactive component (77.6±12.3 % of total immunoreactivity), but ECRA₁₃₅ is detected at a significant extent, accounting for 20.6±9.6 % of total immunoreactivity, whereas ECRA₁₀₀ may not be invariably observed, accounting for 1.8±3.1% of total immunoreactivity. The treatment of MCF-7 cells with YTX (1 μM) also leads to a change in the relative proportions of E-cadherin and related antigens. A 24 hr incubation with this agent, in fact, leads to an almost doubling (190±32%, n=4) of relative total immunoreactivity (Σ), as compared to controls. Under these experimental conditions, ECRA₁₃₅ is undetectable, whereas E-cadherin and ECRA₁₀₀ are present at almost the same level, consituting the 52.0±1.7 and 48.0±1.7 (n=4) of total immunoreactivity, respectively.

Based on the data shown in FIG. 1 it can be summarized that: OA treatment induces the detection of an antigen showing an electrophoretic mobility corresponding to a molecular mass of about 135 kDa (ECRA₁₃₅,) accompanying the native, endogenous E-cadherin (molecular mass about 126 kDa), whereas in the same experimental system, YTX treatment induces a relative increase in an antigen showing an electrophoretic mobility corresponding to a molecular mass of about 100 kDa (ECRA₁₀₀), present at low levels in control cells. These results then suggest that there is a quantitative relation between the levels of E-cadherin, ECRA₁₃₅ and ECRA₁₀₀, on the one hand, and the concentrations of OA and YTX administered to cultured cells, on the other hand, and also show that the qualitative changes in the cellular pool of E-cadherin and related antigens are maintained even in the case MCF-7 cells are treated with both toxins at the same time.

### Example 2

### Quantitative Relationships Between the Levels of E-Cadherin, ECRA₁₃₅ and ECRA₁₀₀, and the Concentrations of OA and YTX Administered to Cultured Cells.

On the basis of the previous findings, experiments have been carried out aimed at the detection of the minimal OA and YTX concentrations which could induce changes in the levels of E-cadherin and related antigens in MCF-7 cells.

The results obtained after cell treatment with OA are reported in FIG. 2 and show that the decrease in the levels of material immunoreactive to the anti-E-cadherin antibody we have employed, normalized for the surface area of culture vessel supplying the sample we have analyzed, can be detected when MCF-7 cells are treated for 20 hr with OA concentrations higher than 10 nM, and it is maximal at concentrations higher than 75 nM. This decrease in relative total immunoreactivity (Σ) is accompanied by a progressive increase in the levels of ECRA₁₃₅, whose detection starts when MCF-7 cells are treated with 20-30 nM OA, and is maximal after treatment with 75 nM OA (FIG. 2). MCF-7 cell treatments with OA concentrations comprised in the same interval lead to a relative decrease in the levels of ECRA₁₀₀, which are minimal at OA concentrations higher than 50 nM. The results reported in FIG. 2 also show that, under the stated experimental conditions, the proportionality in the assay is kept when the equivalent concentrations of OA are comprised between 30-75 nM, corresponding to about 25-60 ng OA/ml.

This type of analysys has been repeated using YTX, and the data (FIG. 3) show that an increase in the material immunoreactive to the anti-E-cadherin antibody, normalized for the surface area of culture vessel supplying the sample we have analyzed, is detectable when MCF-7 cells have been treated for 20 hr with about 0.2 nM YTX, reaching maximal levels at YTX concentrations equal to or higher than 1 nM. The increase in ECRA₁₀₀, accompanied by the reduction in native E-cadherin, appears to be induced by YTX concentrations comprised between 0.2 and 1 nM (FIG. 3), whereas ECRA₁₃₅ has never been detected after MCF-7 cell treatment with YTX alone.

The data reported in FIG. 3 also show that, under the stated experimental conditions, the proportionality in the assay is maintained when the equivalent concentrations of YTX are comprised between 0.2 and 0.5 nM, corresponding to about 240-600 pg YTX/ml.

### Example 3

### Contemporary Measurement of the Effects of OA and YTX in Cultured Cells.

The differences found in the responses induced by OA and YTX, and the possibility to relate their extent to the concentrations of the agent present in the supernatant of the cells, have led to check whether MCF-7 cell treatments with OA and YTX, contemporary present in the culture medium, would result in the detection of changes in the cellular pool of E-cadherin and related antigens similar to those already observed when cells have been treated with the two agents, separately added to our experimental systems.

The results outlined in Example 1 (FIG. 1), showed that extracts obtained from MCF-7 cells which have been treated for 18 hr with OA and YTX, present at the same time in the culture medium at a concentration of 50 nM, contain measurable levels of E-cadherin, ECRA₁₃₅ and ECRA₁₀₀, and that the qualitative alterations in the pool of E-cadherin and related antigens which have been induced by OA and YTX, added alone to cultured cells, are maintained even when MCF-7 cells were treated with the two toxins added contemporary to cultured cells in our experimental system.

In order to identify the dose-response relationship for each of the two classes of toxins, even in the presence of the other one, further experiments have been performed, following the general protocols reported in the preceding examples. MCF-7 cells were then treated for 20 hr with increasing concentrations of each of the two agents, and either in the presence, or in the absence of an effective concentration of the other agent. FIG. 4 reports the data obtained when MCF-7 cells have been treated with increasing concentrations of OA, in the presence or absence of 10 nM YTX, and shows that the increase in the cellular levels of ECRA₁₃₅ induced by OA at concentrations higher than 30 nM is maintained in the presence of 10 nM YTX, but the relative levels of ECRA₁₃₅ are almost halved as compared to those measured in the absence of YTX. The low levels of ECRA₁₀₀ detected in extracts obtained from cells treated with OA (FIG. 2), instead, have not been observed when the cell incubation is carried out in the presence of both OA and 10 nM YTX, which maintains the relative levels of ECRA₁₀₀ between 40 and 50% of total immunoreactivity. This effect is accompanied by a relative decrease in native E-cadherin, whose relative levels are further diminished upon increasing the OA concentrations added together with 10 nM YTX, reaching about 40% of relative total immunoreactivity (Σ) (FIG. 4). Under these experimental conditions, the levels of total immunoreactivity detected in extracts prepared from MCF-7 cells were anyhow progressively decreased after cell treatments with increasing OA concentrations, and the contemporary presence of 10 nM YTX did not appear to maintain the relative increase in immunoreactivity, even at low OA concentrations (FIG. 4).

When these experiments have been repeated to analyse the effect of increasing concentrations of YTX in our experimental system, and in the presence or in the absence of 50 nM OA, we have obtained results in line with previous data (FIG. 5). Thus, it was confirmed that YTX causes a dose-dependent increase in ECRA₁₀₀, whose levels are reduced by the contemporary presence of OA in the culture medium (FIG. 5). It was also confirmed that ECRA₁₃₅ is undetectable in the absence of OA, and that the relative levels of ECRA₁₃₅ decrease upon increasing YTX concentrations (FIG. 5). Furthermore, it was confirmed that the relative levels of E-cadherin, with regard to total immunoreactivity, are progressively decreased by increasing YTX concentrations in the culture medium. When MCF-7 cells were treated in the presence of 50 nM OA, however, this effect was not detected, and the relative levels of E-cadherin were maintained around 70% of total immunoreactivity, independently of the YTX concentrations employed in the treatment. Finally, when cells were treated with YTX in the presence of 50 nM OA, the doubling of the relative total immunoreactivity (Σ) induced by increasing YTX concentrations was not observed (FIG. 5). Under these experimental conditions, in fact, the levels of relative total immunoreactivity (Σ) were maintained at about 20% of those detected in untreated cells, independently of the YTX concentrations present during the treatment with 50 nM OA.

On the basis of these findings, it can be concluded that the present procedure allows the detection of the overall levels of toxic agents belonging to the groups of YTX and DTX contemporary present in the same sample, with a reasonable accuracy, and hence the present process overcomes the limits of those which have been available to measure the abovementioned components in extracts prepared from contaminated material so far.

### Example 4

### Alterations in the Levels of E-Cadherin and Related Antigens in MCF-7 Cells Treated with Crude Mussel Extracts Containing OA and YTX.

Preparation of Crude Extracts from Mussel Hepatopancreas.

Crude extracts have been prepared from uncontaminated blue mussels (Mitylus galloprovincialis) found on the market. The extracts have been prepared according to the official Italian method (G.U. N. 279, Nov. 29, 1995, D. M. Sanità 31 Luglio 1995). The hepatopancreas has been dissected and 25 gr have been homogenized with 125 ml of acetone, in a Potter-Elvejham homogenizer with a teflon pestle. The homogenate was filtered on paper and the residue was re-extracted with 50 ml of acetone and filtration twice. The three extracts were combined and acetone was evaporated at 40° C. The aqueous residue was next extracted with 50 ml of diethyl ether, and the ether solution was collected. The aqueous sample was then re-extracted with 50 ml of diethyl ether twice, and the three ether extracts were combined. Ether has then be evaporated, and the resulting material was the crude extract from mussel hepatopancreas.

Analysis of the Effect Induced by Mussel Extracts Containing OA and YTX on the Levels of E-Cadherin and Related Antigens in MCF-7 Cells . . .

The aim of these experiments was to ascertain whether OA and YTX can induce alterations of E-cadherin and related antigens also when toxins are present in complex matrices, such as crude extracts obtained from edible molluscs, particularly mussels.

To this aim, a crude extract has been prepared. from mussels available on the market, nominally devoid of DSP toxins, which has been spiked by defined quantities of OA and/or YTX. The crude extract, defined here as “original crude extract”, has then been obtained as described in the section of Methods, and has been employed in the experiment as follows. In the first instance, 1 volume of original crude extract was diluted with 5 volumes of ethanol:dimetylsulfoxide (1:1), in order to obtain a solution which could allow an accurate addition of samples to cell cultures, minimising, thereby, the chance of erratic volumes. These latter extracts, here defined as “diluted crude extract”, received the addition of OA and/or YTX at levels comparable to those which are found in samples from contaminated mussels.

The diluted crude extracts were then added with OA and/or YTX, and three more extracts have been obtained, as specified below:

diluted crude extract containing 36 ng of YTX/μl of original crude extract (defined “mussel extract+YTX”);

diluted crude extract containing 125 ng of OA/μl of original crude extract (defined “mussel extract+OA”);

diluted crude extract containing 36 ng of YTX and 125 ng of OA/μl of original crude extract (defined “mussel extract+YTX+OA”).

The FIG. 6 shows the results obtained in MCF-7 cells. The experiment has been carried out by using Petri dishes containing confluent cells in 5 ml of total culture medium. The quantity of mussel extract added to the cells was equivalent to 1.3 μl of original crude extract, containing 50 ng YTX and/or 170 ng OA, as indicated.

The comparison between results obtained with samples from control cells and from cells which have been treated with a mussel extract devoid of toxins (mussel extract, FIG. 6), shows that no qualitative changes in the expression of E-cadherin and related antigens is detectable, as a consequence of cell treatment with extracts prepared from mussels which were not contaminated by DSP toxins (OA and YTX). MCF-7 cell treatment with mussel extracts containing YTX, instead, caused a net increase in ECRA₁₀₀, at levels comparable to those of E-cadherin, leading to an almost doubling of the relative total immunoreactivity (Σ) we have detected (FIG. 6). Cell treatment with extracts containing OA, in turn, led to the detectable appearance of ECRA₁₃₅, accompanied by a net decrease in the relative total immunoreactivity (Σ). Finally, when both toxins were present in the mussel extract employed in the treatment, both an increase in the levels of ECRA₁₀₀ and the appearance of ECRA₁₃₅ were detected (FIG. 6), accompanied by a decrease in the relative total immunoreactivity (Σ).

The alterations of E-cadherin and related antigens induced by OA and YTX, are then detectable even in the case these toxins are present in complex biological matrices, such as the crude extracts prepared from mussel hepatopancreas.

1. A process for the qualitative and quantitative determination of toxins belonging to the group of dinophysistoxins and yessotoxins in a sample, based on the evaluation of the quantity of the E-cadherin protein and of the related antigens, ECRA₁₀₀ and ECRA₁₃₅ in an in vitro cell system treated with said sample.

2. The process according to claim 1, wherein said qualitative determination consists of an observation of the cellular content of the E-cadherin protein and related antigens, ECRA₁₀₀ and ECRA₁₃₅, in the cell system treated with a sample whose contamination has to be measured.

3. The process according to claim 1, wherein said quantitative determination comprises an observation of the changes in the cellular content of the E-cadherin protein and related antigens, ECRA₁₀₀ and ECRA₁₃₅, in the cell system treated with a sample whose contamination has to be measured, with reference to a cell system treated with a control.

4. The process according to claim 1 wherein said evaluation is performed after immunological recognition of E-cadherin and E-cadherin related antigens performed in cell extracts prepared from the in vitro cell system, with anti-E cadherin antibodies.

5. The process according to claim 4 wherein said immunological recognition is performed by techniques chosen in the group consisting of: immunoprecipitation, immunoblotting on a solid phase (Western-blotting), Enzyme Linked Immunosorbent Assay.

6. The process according to claim 5 wherein said technique is an immunoblotting on a solid phase (Western-blotting).

7. The process according to claim 1, wherein said toxins belongs to the group of:
yessotoxins and their derivatives and structurally related analogs and to the group of okadaic acid and its derivatives and structurally related analogs.

8. The process according to claim 7, wherein said yessotoxins are chosen in the group consisting of: yessotoxin, homoyessotoxin, 45-hydroxyyessotoxin, 44-carboxyyessotoxin

9. The process according to claim 7, wherein said okadaic acid structurally related analogs are chosen in the group consisting of: dinophysistoxin 1, dinophysistoxin 2, dinophysistoxin 3.

10. The process according to claims 1-9 further comprising the following steps:
a) preparation of a sample whose contamination has to be evaluated,
b) incubation of the sample in an in vitro cell system,
c) preparation of cytosoluble extracts of the cell system and fractionation of the extracts on the basis of the molecular mass of the proteic components,
d) recognition of the E-cadherin and related antigens, ECRA₁₀₀ and ECRA₁₃₅, by anti-E-cadherin antibodies.

11. The process according to claim 10 wherein said sample is a crude mollusc extract.

12. The process according to claim 11 wherein said crude extract is prepared by extraction and separation with organic solvents.

13. The process according to claim 10 wherein said cell system in step b) of the process is chosen among cell lines expressing the human E-cadherin antigen.

14. The process according to claim 13 wherein said E-cadherin expressing cell lines are MCF-7, A549, BxPc3.

15. The process according to claim 14 wherein the cell line is represented by MCF-7 and the time of incubation in step b) of the process is comprised between 12 and 24 hours.

16. The process according to claim 10 wherein said recognition in step d) of the process is performed by immunoblotting with an anti-E-cadherin antibody.

17. The process according to claim 16 wherein said antibody is monoclonal.

18. The process according to claim 10 wherein said fractionation in step c) of the process is performed by denaturing polyacrylamide gel electrophoresis.

19. The process according to claim 18 wherein said gel is also reducing.

20. The process according to claims 9-19, wherein said recognition in step d) of the process is followed by a step e) of estimation of the levels of immunoreactivity obtained for the antigens: E-cadherin, ECRA₁₀₀ and ECRA₁₃₅.

21. The process according to claim 20 wherein said estimation is carried out by visual inspection.

22. The process according to claim 20 wherein said estimation is carried out by the use of densitometric analyses and calculation of the value of total immunoreactivity and of relative total immunoreactivity (Σ) of the sample.

23. A process for the qualitative and quantitative determination of toxins belonging to the group of dinophysistoxins and yessotoxins, based on the evaluation of the quantity of the E-cadherin protein and of the related antigens, ECRA₁₀₀ and ECRA₁₃₅, in an in vitro cell system, according to claims 1-22 wherein said sample is a sea product.

24. Process according to claim 23 wherein said sea product is foodstuff for human and animal consumption.

25. Process according to claim 24 wherein said product are molluscs.

26. Process according to claim 25 wherein said molluscs are mussels and scallops.

27. Antigen ECRA₁₀₀ immunologically related to E-cadherin, with a molecular mass of 100 kDa.

28. Use of the antigens E-cadherin, ECRA₁₀₀ and ECRA₁₃₅ to detect the presence, to identify the belonging group of a toxin and to measure a toxin level in a sample.

29. Use of the antigens according to claim 28, wherein said toxins belong to the groups of dinophysistoxins and of yessotoxins.

30. Use of the antigens according to claim 29 wherein said toxins are chosen among: yessotoxin, homoyessotoxin, 45-hydroxyyessotoxin, 44-carboxyyessotoxin, dinophysistoxin 1, dinophysistoxin 2, dinophysistoxin 3, okadaic acid, and their derivatives and structurally related analogs.
